(19) United States
(12) Patent Application Publication
(10) Pub. No.: US 2007/0298409 A1
(43) Pub. Date: Dec. 27, 2007

(54) ATP-METRY FOR DETECTING AND COUNTING VIRUSES

(75) Inventor: Dominique CHAMPIAT, Les Matelles (FR)

Correspondence Address: FOLEY & LARDNER LLP, 1530 PAGE MILL ROAD, PALO ALTO, CA, 94304, US

(73) Assignee: Testlife Technologies

(21) Appl. No.: 11/737,049

(22) Filed: Apr. 18, 2007

Related U.S. Application Data
(63) Continuation of application No. PCT/FR05/02596, filed on Oct. 19, 2005.
Application No. PCT/FR05/02596 is a continuation of application No. PCT/FR05/02595, filed on Oct. 19, 2005.

(30) Foreign Application Priority Data
Oct. 19, 2004 (FR) 0411085

(51) Int. Cl. C12Q 1/70

(52) U.S. Cl. 435/5

(57) ABSTRACT

The invention concerns the use of ATP-metry for detecting and counting viruses, via free adenyl nucleotides of their target host cells or via adenyl nucleotides bound to the viral DNA or RNA The invention also concerns the method for determining viruses by ATP-metry.

### FIELD OF THE INVENTION

The present invention relates to a novel technique of ATP-metry for detecting and counting viruses based on adenyl nucleotides (ANs) of the host cells or of the genetic inheritance (DNA or RNA) of the viruses. It also relates to the use of this novel technique and to a method of implementation, firstly using host cells, according to which the viruses to be determined are lytic (at the end of their development, they cleave the wall of the host cells) or nonlytic (at the end of their development, they cross the wall of the host cells without destroying it), or, secondly, using the DNA or the RNA of the viruses depending on whether DNA viruses or RNA viruses are involved.

### PRIOR ART

It is known that ATP-metry, which is based on the reaction:
luciferin+ATP+O₂+Mg²⁺+luciferase→oxyluciferin+photons, (1)
makes it possible to effectively measure the ATP content of a medium. This reaction is specific for ATP, irrespective of the luciferin (substrate)/luciferase (enzyme) system used. It makes it possible to distinguish between dead cells (devoid of ATP) and live cells when the latter contain ATP.

However, ATP-metry is not applicable to viruses as organisms, and so viruses are devoid of free intracellular ATP, ADP and AMP. For their development, viruses use the ATP of their host cells. When they reach maturity, they leave their host cells to go and infect other cells.

Now, it is known that, within the same species or variety of nonviral cells, the total free intracellular AN content is constant, considering relationship (2):
[AN]=[ATP]+[ADP]+[AMP]=Ct, (2)
to this effect, see the article by D. Champiat et al., Luminescence, 2001; 16:193-198, where it proposed, firstly, to convert the AMP and, respectively, the ADP to ATP by means of pyruvate kinase and, respectively, myokinase, and, secondly, to measure the light emitted (in RLU, i.e. in relative light units) without the addition and then after the addition of a further 10 μl of ATP.

It has just been found, surprisingly, that the content of total ANs obtained by cleavage of the viral DNA or RNA is also constant.

It is known, from U.S. Pat. No. 3,575,812 A, that it has already been proposed to detect viruses by ATP-metry by means of the ATP of their host cells. The results obtained are not reproducible as long as the free ATP content of the host cells varies. This is clearly confirmed by an article from 2005, after the priority date of the present invention, see Dick van der Kooij et al., Water Research, 2005; 39: 2789-2798, where it is shown that the ATP content varies by a factor of 1 to 100 (FIG. 2 of said article). As will be seen below, it is necessary to take into account (a) the abovementioned relationship 2 and (b) the content of extracellular ANs, when the virus used is lytic.

It is also known, from U.S. Pat. No. 6,312,902, that it has been proposed to cleave the DNA or RNA of viruses in order to determine their presence in a sample by ATP-metry. Said document neither describes nor suggests, firstly, determining the ANs in order to evaluate the number of viral strains that may be contained in said sample, nor, secondly, using a metered addition.

### OBJECTIVE OF THE INVENTION

There exists a need, which is acutely apparent, with regard to the detection and counting of viruses.

It is therefore envisioned to provide a new technical solution using ATP-metry relating either to all the free ANs of the host cells, expressed in the form of ATP, in order to meet this need, said ANs being free extracellularly when the virus to be tested is lytic and intracellularly when the virus to be tested in nonlytic, or to the ANs produced by cleavage or hydrolysis of the DNA or of the RNA of the virus.

### SUBJECT OF THE INVENTION

According to a first aspect of the invention, the use of bioluminescence according to reaction (1):
luciferin+ATP+O₂+Mg²⁺luciferase→oxyluciferin+photons, (1)
is provided for detecting and counting viruses, said use being characterized in that it implements (a) bringing said viruses into contact with cells of their target in an aqueous liquid medium free of ATP, of ADP and of AMP, and then measuring the content of free adenyl nucleotides (ANs) originating from the cells of the target, expressed in the form of ATP, taking into account the fact that the sum of the free intracellular ATP, ADP and AMP of the same family of cells is constant according to relationship (2):
[AN]=[ASP]+[ADP]+[AMP]=Ct, (2)
after having converted the free ADP and AMP of the target to ATP, or (b) cleaving the viral genetic inheritance, consisting of the DNA or the RNA of said viruses, by means of a DNAse or, respectively, an RNAse, with conversion of the dAMP, dADP and dATP dimers to AMP, ADP and AMP monomers, converting the total AMP and ADP to ATP, and then measuring the content of adenyl nucleotides (ANs) originating from the cleavage of the viral DNA or RNA, expressed in the form of ATP, taking into account the fact that the sum of the ATP, ADP and AMP of the genetic material of the same family of viruses is constant according to said relationship (2),
said measurement being carried out (i) without the addition of ATP and (ii) after the addition of a known amount of ATP.

According to a second aspect of the invention, a method is provided for detecting and counting viruses by ATP-metry based on the ANs of the cells of the target, or on the ANs obtained by cleavage of the viral DNA or RNA, said ANs being expressed in the form of ATP.

According to another aspect of the invention, an assay kit for implementing said method is provided.

Said assay kit is characterized in that it comprises the firefly luciferin/luciferase combination and ATP for the metered addition, and, where appropriate, myokinase, pyruvate kinase, a DNAse, an RNAse and/or pyruvate orthophosphate dikinase and/or microbeads coated with ApoH or with an antibody specific for the virus to be determined.

Said kit may also comprise, where appropriate, live and healthy cells of the target.

Abbreviations

For convenience, the list of abbreviations and acronyms used in the present invention has been provided below.

| | |
|---|---|
| ADP | adenosine diphosphate, |
| AMP | adenosine monophosphate, |
| AN | adenyl nucleotide (other nomenclature that can be |
| | used: adenosine nucleotide), the ANs as a whole |
| | comprise herein ATP, ADP and AMP, |
| ATP | adenosine triphosphate, |
| cAMP | cyclic adenosine monophosphate, |
| dADP | adenosine diphosphate dimer, |
| dAMP | adenosine monophosphate dimer, |
| GDP | guanosine diphosphate, |
| GTP | guanosine triphosphate, |
| NDPK | nucleotide diphosphate kinase, |
| RLU | relative light unit. |

### BRIEF DESCRIPTION OF THE DRAWINGS

FIG. 1 shows the verification of growth by measuring ATP concentration: demonstration of the activity of bacteriophages. a0) Control of growth measured by optical density. a1) Action of bacteriophages measured by optical density. b0) Control of growth measured by assaying ATP. b1) Action of bacteriophages measured by assaying ATP.

FIG. 2 shows the verification of growth by measuring ATP concentration: demonstration of lysogenesis. a0) Control of growth measured by optical density. a1) Action of lysogenesis measured by optical density. b0) Control of growth measured by assaying ATP. b1) Action of lysogenesis measured by assaying ATP.

FIG. 3 shows the verification of growth by measuring ATP concentration: intracellular ATP and Extracellular ATP.

FIG. 4 shows a culture of streptococcal strain M10, phage-inoculated at the beginning of culture with the SM10 phage.

### DETAILED DESCRIPTION OF THE INVENTION

Viruses

Viruses are not cells. They are intracellular parasitic structures. Viruses can only reproduce when they are inside a cell. Reference is then made to virions (viral particles). A virus outside a cell has no metabolic activity. Viruses which parasitize prokaryotic cells (bacteriophages) can be distinguished from those which parasitize eukaryotic cells.

The particularities of viruses are essentially the following. They occupy a “strange undetermined region” between the living and the nonliving. They resemble the living since they have genetic material and are capable of mutations and recombinations. They can therefore evolve and adapt to changing media. However, at the same time, they are acellular: they have neither ribosomes nor metabolic machinery allowing them to synthesize proteins and to generate energy. In the absence of these components, viruses can only reproduce inside host cells and even then, their method of reproduction is unique.

It is known that any cell reproduces by increasing in size, and then dividing into two new cells, each containing a complete set of the components required for life. On the other hand, viruses are disassembled into their components, i.e. into proteins and nucleic acid; the metabolic machinery of the host cell then produces a few tens to a few hundred viral genomes and as many protein capsids which will constitute the new viral envelopes. All these components are then assembled and produce new definitive viral particles.

Viruses do not grow. They do not appear to obey the laws of thermodynamics which apply to open systems.

Moreover, viruses can be crystallized. This is a property of minerals and of most organic molecules, but not of the living cell. When placed under good conditions of moisture and in the presence of living cells, viruses make them produce new viral particles.

Viral Biological Cycles

It is recalled that viruses consist of a nucleic acid and of an envelope. The viral genome consists of a single nucleic acid (DNA or RNA) molecule, which is generally linear or circular, ranging from a few genes to several hundred, which represents a certain number of bases (NMP: AMP, CMP, GMP, TMP in the case of RNA, ribonucleic acid, or dNMP: dAMP, dCMP, dGMP, dTMP in the case of DNA, deoxyribonucleic acid). The nucleic acid is surrounded by a protein shell: the capsid. In certain viruses, this capsid is itself surrounded by a membranous envelope, formed from a double lipid layer containing proteins.

Three types of reproductive cycles are known in viruses: the lytic cycle, the lysogenic cycle and the continuous release cycle. The first two cycles concern almost exclusively bacteriophages. For more than 95% of known phages, the genetic material is a double-stranded DNA molecule from 5 to 650 kbp in size (i.e. a potential of 10 000 to 1 300 000 nucleotide triphosphates), and their size ranges from 24 to 200 nm.

The lytic cycle takes place when a virus invades a cell, reproduces, and then disperses following lysis of the host cell. A cell invaded by a lytic virus is almost invariably killed in a short period of time.

In the lytic cycle, the bacterial chromosome is destroyed. The viral DNA is translated into proteins, those which are necessary for replication of the introduced virus. Replication of the viral DNA takes place. These copies constitute templates for the synthesis of the capsid proteins. This synthesis is performed by the ribosomes of the bacterium (diversion of the bacterial machinery), hence a substantial disturbance in cellular energy and particularly in intracellular ANs since the viruses do not have their own source of energy.

Thus, when nucleic acid synthesis occurs, the nucleotides (bases: ATP, CTP, GTP, TTP, i.e. the XTPs) will be produced by the host cell's machinery. Next, self-assembly of the viral proteins in DNA (or RNA) takes place. The bacterium breaks up and releases numerous virions. This remark is very important in allowing us to differentiate the nucleic acids derived from viruses or from any other prokaryotic or eukaryotic cell, since, before bringing about the hydrolysis of the nucleic acids, the adenyl nucleotides present, which will attest to the presence of nonviral cells, will be assayed.

The lysogenic cycle takes place when a virulent or lytic virus combines its genetic material with that of the host cell and as a result becomes dormant. The DNA (or RNA) of the virus replicates at the same time as that of the host cell, which is referred to as a lysogenic cell; the virus or phage is called a prophage. Certain stimuli lead the prophage to become virulent and to initiate a lytic cycle. The lysogenic cell is then lysed and the viral particles are released.

In the lysogenic cycle, the viral DNA (or RNA) integrates into the bacterial chromosome. The bacterium continues to live until an outside event causes it to switch into the lytic cycle. Certain lysogenic bacteria are extremely important for human health. For example, the bacterium responsible for diphtheria, Corynebacterium diphtheriae, produces the toxin responsible for this disease only if its DNA is infected by a prophage carrying the gene which encodes the diphtheria toxin. The same phenomenon is noted in Clostridium botulinum, responsible for botulism, and in Streptococcus pyrogenes, the agent for scarlet fever.

Therefore, in these cases (lytic cycle or even lysogenic cycle since, during the latter, the virus becomes dormant until an outside event causes it to switch into the lytic cycle), assaying the extracellular ANs (released by lysis of the host cell wall) and total ANs of said host cells will make it possible to demonstrate the presence of virus. The extracellular ANs will allow us to quantify the number of viruses and the ANₑₓₜᵣₐ/ANₜₒₜₐₗ ratio will give us the level of contamination. For example, if ANₑₓₜᵣₐ=ANₜₒₜₐₗ, this implies that all the host cells are lysed and therefore dead (necrosis).

The continuous release cycle is performed by some phages and many animal viruses. These viruses reproduce and are released without interruption by host cells which remain intact. The viruses penetrate the cell by endocytosis.

The endocytic vesicle fuses with a lysosome, thereby allowing release of the viral capsid which can release its genetic material into the cytoplasm of the host cell. The genetic material is replicated and used to produce new capsids which include the viral genetic material so as to form nucleocapsids. The latter are transported by the endoplasmic reticulum and the Golgi apparatus of the host cell to the plasma membrane, where the viral particle buds and is released, surrounded by a membrane envelope originating from the host cell. The flu virus, mumps virus, measles virus or rabies virus have such continuous release cycles.

In this case, assaying the extracellular ANs does not provide any information; on the other hand, as previously pointed out, given the metabolic, and particularly energy-related, disturbance of the host cell in the presence of virus, assaying total ANs will make it possible to detect this anomaly compared with the assaying of the ANs of the same cells devoid of virus.

This sensitivity can be increased by assaying the other nucleotides of the host cell by luminescence, as proposed in the article by M. Gendraud, Physiol. Vég. 1977 15(1) 121-132, according to the mechanism:
XTP+ADP→XDP+ATP in the presence of NDPK.

Thus, according to one aspect of the invention, a method is provided for detecting and counting lytic viruses by ATP-metry based on the ANs extracellularly released by lysis of the wall of the cells of the target, said ANs being expressed in the form of ATP.

According to another aspect of the invention, a method is provided for detecting and counting continuous release viruses by ATP-metry based on the free ANs of the cells of the target, or based on the ANs produced by cleavage of the viral DNA or RNA.

It is thus possible to detect and count the bound total ANs which are associated with the genetic inheritance of the viruses, consisting of their DNA for DNA viruses, or their RNA for RNA viruses, said bound ANs being released by the action of a DNAse or an RNAse, and then, where appropriate, the conversion of the dAMP, dADP and dATP dimers to AMP, ADP and ATP monomers, said AMP and ADP being converted to ATP in order to measure the ANs in the form of ATP.

Method Using Target Cells

The method targeted is a method for detecting and counting the strains of a lytic virus by bioluminescence according to reaction (1):
luciferin+ATP+O₂+Mg²⁺luciferase→oxyluciferin+photons, (1)
said method, which is based on the fact that the sum of the free intracellular adenyl nucleotides (ANs) of the noninfected target is constant according to relationship (2):
[AN]=[ATP]+[ADP]+[AMP]=Ct, (2)

being characterized in that it comprises the following steps consisting in:

| | |
|---|---|
| (1°) | using an aqueous liquid sample (S), which is liable |
| | to contain strains of a virus (V) to be tested and |
| | which is devoid of free ANs; |
| (2°) | bringing said sample (S) into contact with the |
| | cells of the target; |
| (3°) | leaving the resulting reaction medium to incubate |
| | until the virus, reaching the end of its |
| | development, lyses the wall of the target; |
| (4°) | recovering the resulting extracellular medium and |
| | treating it in order to convert the ADP and AMP to |
| | ATP; |
| (5°) | introducing, into the resulting medium of step |
| | (4°), a luciferin and a luciferase, first (i) |
| | without the addition of ATP, and then (ii) after |
| | the addition of a known amount of ATP; |
| (6°) | measuring the amplified signal of the light emitted |
| | by reaction (1) without the addition of ATP, and |
| | then after the addition of a known amount of ATP; |
| | and |
| (7°) | determining the content of extracellularly free |
| | total ANs in the form of ATP, and then deducing |
| | therefrom the number of strains of said virus (V) |
| | in the initial sample (S). |

For the deduction of the number of strains of said virus, step (7°) can comprise the use of a pre-established system of graphs. As a variant, the content of extracellular total ANs can be determined by comparison with that of the cells of the target obtained by reproducing of steps (1°) to (7°) in the absence of said virus, and then deducing therefrom the number of strains of said virus (V) in the initial sample (S). In practice, the metered addition technique allows a good direct determination of the number of strains of the virus.

Also targeted is a method for detecting and counting the strains of a continuous release virus by bioluminescence according to reaction (1):
luciferin+ATP+O₂+M+luciferase→oxyluciferin+photons, (1)
said method, which is based on the fact that the sum of the free intracellular adenyl nucleotides (ANs) of the noninfected target is constant according to relationship (2):
[AN]=[ATP]+[ADP]+[AMP]=Ct, (2)

being characterized in that it comprises the following steps consisting in:

| | |
|---|---|
| (1a°) | using an aqueous liquid sample (S), which is liable |
| | to contain strains of a virus (V) to be tested and |
| | which is devoid of free ANs; |
| (2a°) | bringing said sample (S) into contact with the |
| | cells of the target; |
| (3a°) | leaving the resulting reaction medium to incubate |
| | until the virus, reaching the end of its |
| | development, crosses the wall of the target; |
| (4a°) | recovering the cells of the target, subjecting them |
| | to lysis so as to recover the resulting |
| | extracellular medium, and then treating said |
| | intracellular medium in order to convert the ADP |
| | and AMP to ATP; |
| (5a°) | introducing, into the resulting medium of step |
| | (4a°), a luciferin and a luciferase, first (i) |
| | without the addition of ATP, and then (ii) after |
| | the addition of a known amount of ATP; |
| (6a°) | measuring the amplified signal of the light emitted |
| | by reaction (1) without the addition of ATP, and |
| | then after the addition of a known amount of ATP; |
| | and |
| (7a°) | determining the content of intracellularly free |
| | total ANs, in the form of ATP, and deducing |
| | therefrom the number of strains of said virus (V) |
| | in said sample (S). |

Step (7a) can be carried out as indicated above for step (7°); in particular, by comparison with the cells of the target treated by reproducing steps (1a°) to (7a°) in the absence of said virus. As a variant, the content of intracellular total ANs can be determined by means of a pre-established system of graphs. The number of strains of said virus (V) in said sample (S) is then deduced therefrom.

Briefly, steps (1°)-(2°) and (5°) are identical to steps (1a°)-(2a°) and (5a°); steps (3°) and (6°)-(7°) are substantially analogous to steps (3a°) and (6a°)-(7a°); and step (4°) is different than step (4a°). The methods of the implementation of the identical or similar steps will be treated in common. On the other hand, those of the respective steps (4°) and (4a°) will be treated separately.

The sample (S) is an aqueous liquid composition; where appropriate, it can be an organic liquid composition. This sample (S) comes from a gaseous sample taken [in particular by sparging], a solid sample taken [in particular by contact, dissolution or dispersion] or a liquid sample taken [by extraction, dissolution or emulsion] by means of a liquid which is advantageously aqueous.

The abovementioned reaction (1) provides oxyluciferin, photons, AMP and one or more phosphates, mainly pyrophosphate. It is specific for ATP, the luciferin and the luciferase being at an optimal concentration, and the number of photons emitted once these three substances are present together is directly proportional to the amount of ATP. In the organism and any reaction medium, extracellular ATP disappears relatively rapidly, either through reuse, or mainly by degradation.

ATP is involved in the cell as a source of energy (mechanical energy, osmotic energy, chemical energy, caloric energy, light energy), phosphate donor, pyrophosphate donor, AMP donor and adenosine donor.

The ATP content in cells from the same species varies greatly depending on the physiological state. The detection threshold is of the order of 10³ bacteria. According to the invention, a better sensitivity will be achieved, ranging from 1 attomole of ATP (without stabilization of the light signal emitted) to 0.5 attomole of ATP (with stabilization of said signal), which corresponds approximately to the average content of total intracellular ANs of a bacterium.

When considering relationship (2), the intracellular content of cyclic adenosine monophosphate (cAMP), which is the precursor involved in the synthesis of AMP, is ignored here. This content is virtually negligible in the reaction media of the invention.

According to the invention, the well-known principle of the firefly (Photinus pyralis), which operates with an enzyme (luciferase), a luminiferous substrate (luciferin) and a coenzyme (in this case ATP), is used. The result is often displayed on a photometer (or luminometer) in RLU, which, although proportional to the amount of ATP does not make it possible to determine from one sample to another the real concentration of ATP.

In order to overcome this difficulty, the introduction of a known amount (for example, 10² to 10 pmol of ATP), after the first reading (undertaken without the introduction of ATP), is recommended. However, the “metered” introduction technique does not allow a quantitative determination of the count since the ATP content in said cells does not remain constant: there is a rapid turnover depending on the physiological state.

On the other hand, the cells of a given target all have the same AN content. According to the invention, by determining the content of AN, expressed in the form of ATP, it will be possible to carry out quantitative determinations for counting the cells of the target and the strains of virus.

Advantageously, step (1°) or (1a°) of the method of the invention, which relates to isolation and concentration, is carried out by
membrane filtration,
evaporation-centrifugation, in particular under vacuum and at ambient temperature (15-25° C.), and/or
immunocapture.

The immunocapture technique is preferred. It makes it possible to concentrate and purify the strains of the virus by binding them with antibodies. Practically, these antibodies can be directed against surface antigens of the virus without destroying it. Also practically, these antibodies are immobilized on microbeads of magnetic latex for the purpose of concentrating and purifying the virus-antibody-bead-type conjugation products in a magnetic field and recovering said conjugation products. As a variant, nonmagnetic or nonmagnetizable microbeads, attached to the antibodies which bind the strains of the virus, also make it possible to concentrate and purify said strains by settling out.

The best method for implementing the immunocapture consists in using magnetic microbeads coated with ApoH, a protein which binds living matter and does not bind fragments, in particular the wall, of dead organisms. It is recalled that ApoH is a protein which only manifests itself just before or at the time of cell death (apoptosis).

Said conjugation products are then separated, if necessary, in particular by elution, in order to have a concentrated liquid composition of viral strains which are no longer bound to the antibodies.

Where appropriate, in order to limit the dilutions which decrease the sensitivity, it may be judicious to concentrate said liquid composition by means of an evaporation-centrifugation device (operating at from 2000-10 000 revs/15 minutes to 2000-10 000 revs/minute), which makes it possible to dry a large number of samples or reaction media in a few minutes, without any loss of products. Evaporation-centrifugation at ambient temperature offers the advantage of being able to remove most of the water from the medium containing the strains.

Also advantageously, steps (2°)-(3°) or (2a°)-(3a°), related to bringing the viral strains into contact with the strains of the target and then to their development, are carried out according to a method known to those skilled in the art.

In step (4a°), the lysis of the cell wall of the target is carried out in the medium resulting from step (3a°) addition of an aqueous buffer containing
(i) Tris plus EDTA, and/or
(ii) DMSO,
   and then treatment in a microwave (for approximately 1 minute) in order to open up the cells of the target, followed by rapid cooling (in particular in a refrigerator) and, if necessary, centrifugation so as to recover the resulting liquid medium. The lysis is required in order to be able to gain access to the intracellular ANs.

The conversion, in step (4°) or (4a°), of the ADP and AMP to ATP is carried out by means of myokinase and pyruvate kinase. The reaction mechanisms are the following:

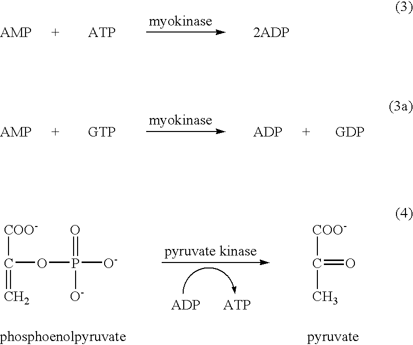

In order to gain time, step (5°) of the method of the invention, relating to the conversion of the ADP and AMP to ATP, can be implemented at the same time as step (4°).

Advantageously, step (5°) or (5a°) of the method of the invention is implemented with firefly (Photinus pyralis) luciferin and luciferase. The substrate and the enzyme can be extracted simultaneously from the firefly.

In practice, it is recommended that step (6°) or (6a°), relating to the measurement of the light emitted by reaction (1), be carried out in the presence of a substance that stabilizes the emission of photons, at a value that is substantially constant for at least 10 minutes. Among the substances which are suitable for this purpose, mention may be made of:
pyruvate orthophosphate dikinase (PPDK), which converts AMP and pyrophosphate, produced during the abovementioned reaction (1), to ATP, and
adenosine phosphate deaminase, which degrades the residual ADP and/or AMP that may be present in the reaction medium.

The first enzyme provides a stable signal by regenerating ATP in a substantially continuous manner. The second enzyme makes it possible to reduce the background noise due to the residual presence in the assay medium of ADP and/or AMP, without the process of using ATP as a light energy source being disturbed.

Said second enzyme, adenosine phosphate deaminase, is more advantageously used to eliminate the nucleotide residues in the reaction medium, and more particularly to remove, by destroying them, the extracellular nucleotide residues present, where appropriate, in the sample during the abovementioned step (1°).

In practice, to stabilize the emission of photons in accordance with reaction (1), it is more particularly recommended to use PPDK in step (5°).

The method of the invention is particularly suitable for detecting and counting viruses, such as HIV, bacteriophages or the hepatitis C virus.

The ANs, initially bound and thus obtained, are converted to ATP by means of myokinase and of pyruvate kinase as in step (4°).

The emission of photons is implemented by means of firefly luciferin and firefly luciferase as indicated above for implementing step (5°) or (5a°) without the addition of ATP and then after the metered addition of ATP. The firefly luciferin/luciferase combination is commercially available, in particular from the company known as Controlife.

Advantageously, the process is carried out as indicated above, with said emission of photons being stabilized by means of PPDK.

The measurement of the amplified signal and the determination are carried out as indicated above in steps (6°) and (7°).

According to the invention, a method is recommended for detecting and counting viruses by ATP-metry, said method being characterized in that it comprises:
cleaving the viral DNA or RNA by means of DNAse or RNAse, and then, where appropriate, converting the dAMP and dADP dimers to AMP and ADP monomers,
converting the AMP and ADP thus obtained to ATP,
bringing the ATP into contact with a luciferin and a luciferase, first
(i) without the addition of ATP, and then (ii) after the addition of a known amount of ATP,
measuring the amplified signal of light emitted by reaction (1) without the addition of ATP, and then after the addition of ATP, and
determining the content of total ANs initially bound, expressed in the form of ATP, so as to deduce therefrom the number of viruses.

Method Using the Genetic Inheritance of the Viruses

This method will now be described succinctly with reference to the above steps.

An aqueous liquid sample (S) is used as in step (1°) above. The isolation/concentration is carried out by
membrane filtration,
evaporation-centrifugation, in particular under vacuum and at ambient temperature (15-25° C.), and/or
immunocapture,
   as indicated above, advantageously by means of ApoH.

The viral wall is lysed by addition of an aqueous buffer containing
(i) Tris plus EDTA, and/or
(ii) DMSO,
   and then treatment in a microwave (for approximately 1 minute) in order to open up the cells of the target, followed by rapid cooling (in particular in a refrigerator) and, if necessary, centrifugation so as to recover the resulting liquid medium.

The viral DNA or RNA is cleaved by means (i) of endonucleases in order to release oligonucleotides, for example DNAse or RNAse (in particular RNAse T1 for cleavage of G on the 5′ side, or pancreatic RNAse for cleavage of C/U on the 5′ side), or alternatively restriction enzymes, (ii) of exonuclease, or (iii) of bovine spleen phosphodiesterase (for cleaving RNA from 5′-OH, releasing 3′-Ps) or of snake venom phospho-diesterase (for cleaving RNA from 3′-OH, releasing 5′-Ps) and the dAMP, dADP and dATP dimers present are converted to AMP, ADP and ATP monomers.

For converting the nucleotide monophosphates to nucleoside triphosphates, the operating mechanisms described in U.S. Pat. No. 6,312,902 B can be used.

The preferred mechanisms according to the invention comprise the following:

I

(a) According to ANₙ+PPi→ANₙ₋₁+XTP XTP

under the action:
of DNA polymerase reverse transcriptase for DNA, of RNA polymerase for RNA.

Several enzymes have been used in this conversion, in particular:
AMV reverse transcriptase, MMLV reverse transcriptase, DNA polymerase α and β, Taq polymerase, T4 DNA polymerase, Klenow fragment, poly A polymerase, at concentrations of from 0.1 to 100 U/ml.

(b)→With NDPK, according to: 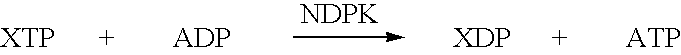
with 0.01 to 0.50 μM, preferably 0.5 μM of ADP and 0.1 to 10 U/ml of NDPK.

II

(a) An AN hydrolysis step, according to:
ANₙ+H₂O→ANₙ₋₁+XMP
ANₙ+exonuclease→nXMP
[i.e. NMP if RNA, or dNMP if DNA].

(b)→With PRPP synthetase, according to: 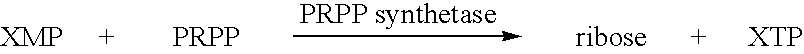
[XTP is either NTP if RNA, or dNTP if DNA].

5-Phosphoribosyl pyrophosphate synthetase (or ribose phosphate pyrophosphokinase) is an enzyme common to various synthetic pathways, including purine synthesis and pyrimidine synthesis in humans. It catalyzes a reaction to transfer pyrophosphate to α-D-ribose derived from the pentose-phosphate pathway. It is advantageously used at a concentration of from 0.001 to 10 U/ml. ATP is the pyrophosphate-donor and energy-donor coenzyme.

5-Phosphoribosylpyrophosphate (i.e. 5-PRPP) is a metabolic “crossroads”, involved as a substrate for many enzymes for the synthesis of nucleotides.
adenine phosphoribosyl transferase
hypoxanthine-guanine phosphoribosyl transferase (HGPRT-ase)
orotate phosphoribosyl transferase (complex U)
5-PRPP Amidotransferase.

The enzyme is inhibited by purine nucleotides: ADP and GDP.

(c) With NDKP, according to: 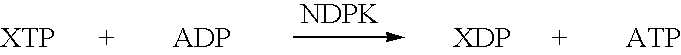
with 0.01 to 0.50 μM, preferably 0.5 μM of ADP, and from 0.1 to 100 U/ml of NDPK (when NDPK is used, the phosphate donor is dCTP or α,β-methyleneadenosine 5′-triphosphate). III With nucleotide monophosphate kinase (NMPK) and pyruvate kinase, according to: 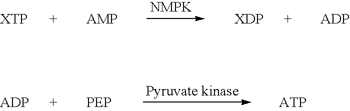
or else with NMPK and/or myokinase.

The latter technique is that which is recommended according to the invention. The limiting and tricky step is the nucleic acid hydrolysis step. The detection threshold depends on the base number composition of the viral genome.

Given that the molecular weight of ATP is 551 and that the ATP detection threshold depends on the luminometer and on the reagent, but that today it is of the order of an attomole, using Avogadro's number (6.022×10²³), it is easy to predict the number of detectable viral particles, the assaying by ATP-metry being carried out with the firefly luciferase/luciferin pair (product is manufactured by the company known as Controlife).

Other advantages and characteristics of the invention will be understood more clearly on reading the following implementation examples. Of course, these examples are not limiting, but are provided by way of illustration.

### EXAMPLE 1

### Protocol of ATP-Metry on Viral DNA or RNA

100 ml of sample buffered with 50 mM Tris HCl, 150 mM NaCl at pH 7.6,
10 μl of ready-to-use beads (with ApoH or antibodies specific for the virus being sought) are added.
Incubation is carried out at 37° C. for 30 to 90 min with moderate rotary agitation.
Washing is performed once in PBS.
The beads are recovered with a magnetized system.
The viruses are lyzed by placing the beads in contact with a DMSO-type extracting solution.
The nucleic acids are then hydrolyzed to nucleotides and converted to ATP.

Provide for:
buffer solution: limits variations in pH of the medium, thereby preventing denaturation of the DNA (or RNA).
SDS (sodium dodecyl sulfate): detergent which cleaves the lipid layers.
EDTA (ethylenediaminetetraacetate): calcium ion chelator. Ca²⁺ ions are necessary for the activity of DNAses, enzymes which destroy DNA and are present in all cells.
Centrifugation for rapid separation of the solid particles (the heaviest) from the substances in solution (less heavy).
Cold sodium acetate and ice-cold ethanol for precipitating the DNA.

### EXAMPLE 2

### Assaying of Bacteriophages

Lactic acid bacteria were used as target cells for bacteriophages which are lytic viruses.

In dairies and cheese dairies, the quality of the dairy products depends on the amount of lactic acid produced by the lactic acid bacteria, and it is advisable to monitor the bacteriophage content in order to prevent the destruction of said lactic acid bacteria.

Increasing amounts of bacteriophages (0 virus/L, 1 virus/L, 10 viruses/L, 20 viruses/L) were added to a known population of lactic acid bacteria [determined according to the method described in the patent application entitled: “ATP-metry based on intracellular adenyl nucleotides for detecting and counting cells, use and implementing method for determining bacteria in particular devoid of ATP” filed on the same day as the present application]. For implementing the above-mentioned steps (1°)-(7°), the correct starting populations are found (0 virus/L, 1 virus/L, 10 viruses/L and 20 viruses/L).

### EXAMPLE 3

### Detection of Bacteriophages by Assaying the NAs of the Host Cells

(a) The ATP, ADP and AMP nucleotides are assayed by bioluminescence in the presence of the luciferin-luciferase complex. Luciferase is an enzyme extracted from the abdomen of the firefly (Photinus pyralis or Luciola mingrelica). One molecule of luciferin is oxidized by 1 mol of ATP and 1 mol of O₂ and, for each luciferin molecule, a quantum of light energy is emitted according to the abovementioned overall reaction (1).

The ATP can be assayed directly by bioluminescence. Here, it is necessary to convert the ADP and the AMP to ATP by means of enzymatic reactions, using myokinase and pyruvate kinase according to the abovementioned reactions (3), (3a) and (4).

### Materials and Methods

Bacterial strains: two industrial strains of mesophilic streptococci, SM10 and SM26, were used for setting up the detection of virulent phages (respectively, Φ10 and Φ26); for the detection of temperate phages, the lysogenic SM16 strain was selected.

Culture medium: the synthetic medium M17 was used in all the assays.

Action of a virulent phage: growth is monitored in 250 ml Erlenmeyer flasks containing 100 ml of M17 medium. The Erlenmeyers are inoculated at 1% w/v with a culture 16 hours old, and incubated at 320C. When the optical density (measured at 580 nm) reaches 0.1, 1 ml of the solution containing the virulent phages (10⁹ pfu/ml) is added.

Action of a temperate phage: the induction of the prophage integrated into the DNA is triggered by the action of mitomycin (MC) at 0.3 μg/ml as soon as the growth reaches an optical density of 0.1.

Measurement of growth: the growth is monitored in parallel by measuring the optical density and by assaying the adenyl nucleotides.

Three samples are thus prepared: No. 1−assaying of total nucleotides−200 μl of sample+1.8 ml of pure DMSO+10 ml Tris buffer (20 mM Tris, 10 mM Mg acetate, pH 7.75); No. 2−assaying of extracellular nucleotides−200 μl of sample, filtered through a Millipore membrane (0.22 μm)+4.8 ml Tris buffer 1; No. 3−assaying of nucleotides with neither extraction nor filtration−200 μl of sample+4.8 ml of Tris buffer 1.

100 μl of luciferase are added to 200 μl of prepared sample, as are:
for assaying ATP, 50 μl of Tris buffer 2 (20 mM Tris, 10 mM Mg acetate, 5 mM K acetate, 40 μM PEP),
for preparing an internal control, 50 μl of Tris buffer 2 containing 10 picomoles of ATP,
for assaying ATP and ADP, 50 μl of Tris buffer 2 containing 2 U of pyruvate kinase, and
for assaying ATP, ADP and AMP, 50 μl of Tris buffer 2 containing 2 U of pyruvate kinase and 2 U of myokinase.

### Results

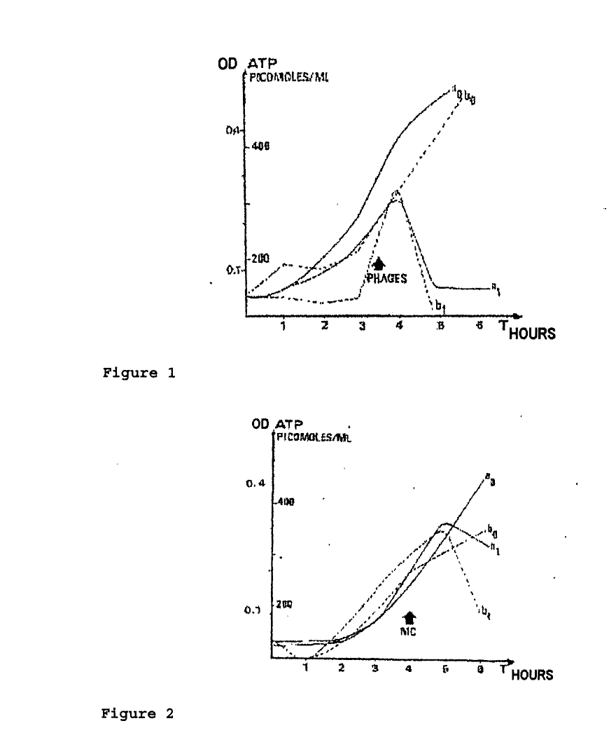

According to FIGS. 1 and 2, the growth curves obtained by assaying ATP are equivalent to those obtained by measuring the optical density. In the two cases (action of bacteriophages and lysis of bacteria after addition of mitomycin), the decrease in growth due to cell lysis is more accentuated in the ATP measurements than when measuring optical density (curves a1 and b1, FIGS. 1 and 2). The ATP concentration gives a better reflection of the amount of “live” bacteria, whereas the optical density measurement also includes the cell debris present in the medium in a considerable amount during the lysis phenomena.

The results reported in FIGS. 1 and 2 correspond to the measurements of total ATP (by extraction of intracellular ATP with DMSO). The arrows indicate the moment at which the phage was introduced.

As indicated in U.S. Pat. No. 3,575,812 A mentioned above, only the RLUs for total (intra- and extracellular) ATP are assayed, without using metered addition. Admittedly, it demonstrates a variation in RLUs in the cells attacked by the phages, but, in the examples of this US patent, the values are sometimes increasing values and other times decreasing values. This is due to the fact that an internal control is not used and the intracellular ATP is not distinguished from the extracellular ATP.

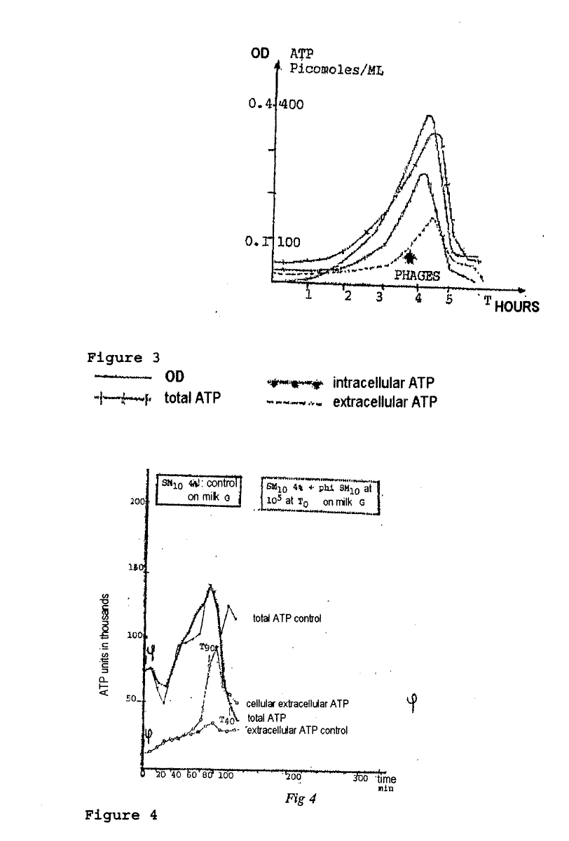

The results of FIG. 3 given herein after make it possible to compare the assays of extracellular ATP compared with total ATP.

The advantage of assaying extracellular NAs lies in the simplification of the protocol: there is no longer any need to lyse the cells (this lysis is performed by the phages). The curves for extracellular ANs after filtration and without filtration are identical (they overlap).

(b) FIG. 4 shows a culture of streptococcal strain M10, phage-inoculated at the beginning of culture with the SM10 phage.

Example 3 shows, in its three variants, that direct assaying of extracellular ANs allows continuous verification to be performed. In fact, this verification can be carried out by automatically taking a sample, then converting the ANs to ATP, and then assaying the ATP thus formed by bioluminescence with an instantaneous reading.

The results obtained according to the three variants of example 3 were obtained from assays carried out on the M17 synthetic reference medium. Now, in a natural medium, it is difficult to determine the optical density and, in addition, the other verification techniques are either laborious or imprecise for rapidly detecting the action of a virus, regardless of whether it is “virulent” or “temperate”. On the other hand, the assaying of NAs is rapid and the use of the internal control consisting of a known ATP solution makes it possible to perform quantification.

The ANₑₓₜᵣₐ/ANₜₒₜₐₗ ratio makes it possible to demonstrate a viral attack and the degree of said attack. It is thus possible to screen up to 10 phages per ml.

The assaying of extracellular ANs, as regards lytic viruses, allows a more rapid and simpler detection. It is the only method which makes it possible to detect a viral attack in a complex medium containing various viral species, by bringing this sample into contact with various cultures of host cells. According to the invention, the sensitivity is increased by assaying the ANs and not the RLUs for ATP.

1. The use of bioluminescence according to reaction (1):
luciferin+ATP+O₂+Mg²⁺+luciferase→oxyluciferin+photons, (1)
for detecting and counting viruses, said use being characterized in that it implements (a) bringing said viruses into contact with cells of their target in an aqueous liquid medium free of ATP, of ADP and of AMP, and then measuring the content of free adenyl nucleotides (ANs) originating from the cells of the target, expressed in the form of ATP, taking into account the fact that the sum of the free intracellular ATP, ADP and AMP of the same family of cells is constant according to relationship (2):
[AN]=[ATP]+[ADP]+[AMP]=Ct, (2)
after having converted the ADP and AMP of the target to ATP, or (b) cleaving the viral genetic inheritance, consisting of the DNA or the RNA of said viruses, by means of a DNAse or, respectively, an RNAse, with conversion of the dAMP and dADP dimers to AMP and ADP monomers, converting the total AMP and ADP to ATP, and then measuring the content of adenyl nucleotides (ANs) originating from the cleavage of the viral DNA or RNA, expressed in the form of ATP, taking into account the fact that the sum of the ATP, ADP and AMP of the genetic material of the same family of viruses is constant according to said relationship (2), said measurement being carried out (i) without the addition of ATP and (ii) after the addition of a known amount of ATP.

2. The use as claimed in claim 1, characterized in that the ATP-metry of lytic viruses is carried out based on the ANs extracellularly released by lysis of the wall of the cells of the target, said ANs being expressed in the form of ATP.

3. The use as claimed in claim 1, characterized in that the ATP-metry of continuous release viruses is carried out based on the intracellular ANs released by causing lysis of the wall of the cells of the target, said ANs being expressed in the form of ATP.

4. A method for detecting and counting lytic viruses by ATP-metry, characterized in that it comprises measuring the ANs extracellularly released by lysis of the wall of the cells of the target, said ANs being expressed in the form of ATP.

5. A method for detecting and counting continuous release viruses by ATP-metry, characterized in that it comprises measuring the free intracellular ANs of the cells of the target, said ANs being expressed in the form of ATP, the content of total free intracellular ANs of the infected cells of the target being different than those of the same cells of said target that are not infected.

6. The method as claimed in claim 4, for detecting and counting the strains of a lytic virus by bioluminescence according to reaction (1):
luciferin+ATP+O₂+Mg²⁺+luciferase→oxyluciferin+photons, (1)
said method, which is based on the fact that the sum of the intracellular adenyl nucleotides (ANs) of the noninfected target is constant according to relationship (2):
[AN]=[ATP]+[ADP]+[AMP]=Ct, (2)
being characterized in that it comprises the following steps consisting of:
(1°) using an aqueous liquid sample (S), which is liable to contain strains of a virus (V) to be tested and which is devoid of free ANs;
(2°) bringing said sample (S) into contact with the cells of the target;
(3°) leaving the resulting reaction medium to incubate until the virus, reaching the end of its development, lyses the wall of the target;
(4°) recovering the resulting extracellular medium and treating it in order to convert the ADP and AMP to ATP;
(5°) introducing, into the resulting medium of step (4°), a luciferin and a luciferase, first (i) without the addition of ATP, and then (ii) after the addition of a known amount of ATP;
(6°) measuring the amplified signal of the light emitted by reaction (1) without the addition of ATP, and then after the addition of a known amount of ATP;
and
(7°) determining the content of extracellularly free total ANs in the form of ATP, and then deducing therefrom the number of strains of said virus (V) in the initial sample (S).

7. The method as claimed in claim 6, characterized in that, in step (7°), the content of extracellular total ANs is determined by means of graphs or by comparison with that of the cells of the target obtained by reproducing steps (1°) to (7°) in the absence of said virus, in order to deduce therefrom the number of strains of said virus (V) in the initial sample (S).

8. The method as claimed in claim 5, for detecting and counting the strains of a nonlytic virus by bioluminescence according to reaction (1) which follows:
luciferin+ATP+O₂+Mg²⁺+luciferase→oxyluciferin+photons, (1)
said method, which is based on the fact that the sum of the intracellular adenyl nucleotides (ANs) of the noninfected target is constant according to relationship (2):
[AN]=[ATP]+[ADP]+[AMP]=Ct, (2)
being characterized in that it comprises the following steps consisting of:
(1a°) using an aqueous liquid sample (S), which is liable to contain strains of a virus (V) to be tested and which is devoid of ANs;
(2a°) bringing said sample (S) into contact with the cells of the target;
(3a°) leaving the resulting reaction medium to incubate until the virus, reaching the end of its development, crosses the wall of the target;
(4a°) recovering the cells of the target, subjecting them to lysis so as to recover the resulting intracellular medium, and then treating said intracellular medium in order to convert the ADP and AMP to ATP;
(5a°) introducing, into the resulting medium of step (4a°), a luciferin and a luciferase, first (i) without the addition of ATP, and then (ii) after the addition of a known amount of ATP;
(6a°) measuring the amplified signal of the light emitted by reaction (1) without the addition of ATP, and then after the addition of a known amount of ATP;
and
(7a°) determining the content of intracellularly free total ANs, in the form of ATP, and deducing therefrom the number of strains of said virus (V) in the initial sample (S).

9. The method as claimed in claim 8, characterized in that, in step (7a°), the content of intracellular total ANs is determined by means of a pre-established system of graphs, or by comparison with that of the cells of the target obtained by reproducing steps (1a°) to (7a°) in the absence of said virus, in order to deduce therefrom the number of viruses (V) of the initial sample (S).

10. The method as claimed in claim 8, characterized in that, in step (4a°), the lysis of the cell wall of the target is carried out in the medium resulting from step (3a°) by addition of an aqueous buffer containing
(i) Tris plus EDTA, and/or
(ii) DMSO,
and then treatment in a microwave (for approximately 1 minute) in order to open up the cells of the target, followed by rapid cooling (in particular in a refrigerator) and, if necessary, centrifugation so as to recover the resulting liquid medium.

11. The method as claimed in claim 6 or 8, characterized in that step (4°) or (4a°) for converting the ADP and AMP to ATP is carried out by means of myokinase and pyruvate kinase.

12. The method as claimed in claim 4 or 5, characterized in that it comprises a phase of capturing the viruses or their host cells by means of magnetic microbeads coated with ApoH.

13. An assay kit for implementing the method as claimed in claim 4 or 5, characterized in that it comprises the firefly luciferin/luciferase combination and ATP for the metered addition, and, where appropriate, myokinase, pyruvate kinase, pyruvate orthophosphate dikinase and/or magnetic microbeads coated with ApoH.

14. A method for detecting and counting viruses by ATP-metry, said method being characterized in that it comprises:
cleaving the viral DNA or RNA by means of DNAse or RNAse, and then, where appropriate, converting the dAMP and dADP dimers to AMP and ADP monomers,
converting the AMP and ADP thus obtained to ATP,
bringing the ATP into contact with a luciferin and a luciferase, first
(i) without the addition of ATP, and then (ii) after the addition of a known amount of ATP,
measuring the amplified signal of light emitted by reaction (1) without the addition of ATP, and then after the addition of ATP, and
determining the content of total ANs initially bound, expressed in the form of ATP, so as to deduce therefrom the number of viruses.
